(19) United States
(12) Patent Application Publication
(10) Pub. No.: US 2004/0127827 A1
(43) Pub. Date: Jul. 1, 2004

(54) Limb injury protectors

(76) Inventor: Rebecca Ann Treadway Fancher, West Monroe, LA (US)

Correspondence Address: Rebecca Ann Treadway Fancher, 907 Warren Drive, West Monroe, LA, 71291, US

(21) Appl. No.: 10/331,547

(22) Filed: Dec. 31, 2002

(51) Int. Cl. A61F 5/00

(52) U.S. Cl. 602/41

(57) ABSTRACT

Limb protectors are disposable, pliable, moisture-resistant plastic coverings which enclose the hand/arm or foot/leg areas and seal off the area with an elasticized opening. These products may be used for covering limb injury sites, such as casts, sutures, burns, IVs, cuts, surgical sites, rashes or other wounds which would require the location of concern to be kept free from moisture or other contaminants. These products may also be used by individuals needing added protection in situations involving harmful or questionable fluids or contaminants, such as medical or emergency personnel and others dealing with body fluids as an occupational hazard, or by individuals seeking added protection in any circumstance to keep the area of concern clean and dry. The uses for these products are many and varied, which will make this an item of demand on the open market.

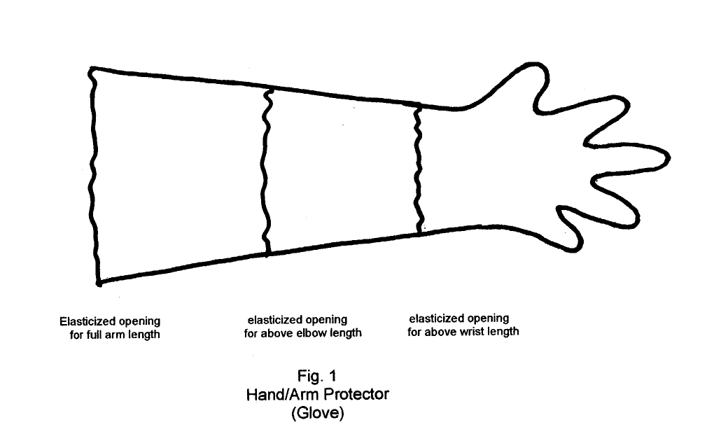

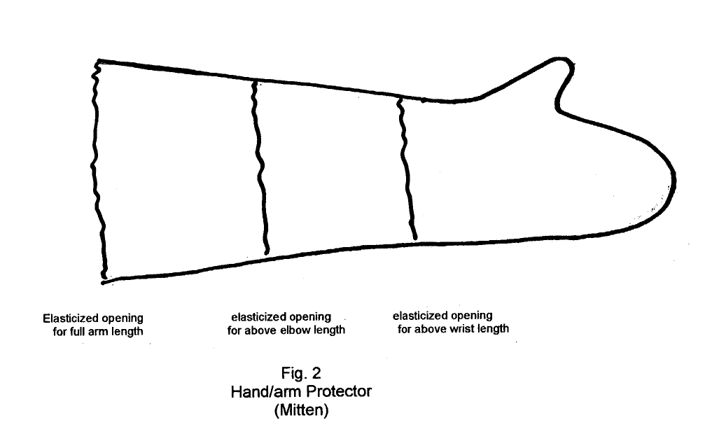

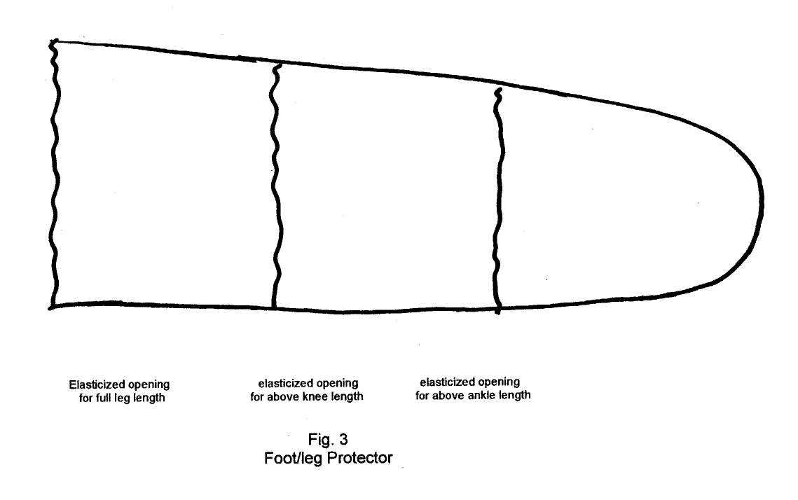

Limb injury protectors are disposable, flexible, water resistent clear plastic coverings which enclose the hand/arm or foot/leg areas and seal off the area with an elastic opening, making the product a perfect covering for casts or sites of medical concern. This product is invented by Rebecca Ann Treadway Fancher, citizen of the United States of America, 907 Warren Drive, West Monroe, La. 71291.

Limb injury protectors are designed to aid individuals with limb injuries to carry on a more normal lifestyle during their time of convelescense while keeping the injury site clean, dry and protected from moisture and contaminants, to help promote proper and desired healing. These protectors are primarily for, but not limited to, medical purposes used as an aid to to perpetuate healing by keeping the targeted area dry, while permitting the individual wearing the protector to comfortably perform normal daily activities such as bathing, washing dishes, going out in the rain, etc. or from airborne pollutants or other contaminants.

The protectors will be manufactured by use of a heated die which will cut and seal the product from a doubled, clear plastic polyethylene or polypropylene film or other appropriate plastic. Elastic will be attached (sewn, glued or heated) at the open edge, similar to the elastic used in plastic shower caps or hair bleaching caps. This product will be made similarly to existing plastic glove products, primarily those used in veterinary medicine, only with an elasticized opening to seal off the targeted area.

This particular plastic is chosen to allow the wearer the comfort of natural ‘feeling’ to the elements, which is important for bathing, etc., and will easily slip over the problem area without sticking or pulling, (such as do the current medical latex gloves) while providing a fit similar to the limb (as opposed to attaching a cumbersome plastic grocery or garbage bag to attempt the same purpose) and providing an elasticized snug fit to efficiently seal off the area to be protected. The fingers of the glove for the hand/arm protector will allow the individual the independence and satisfaction of being able to make the limb useful for washing, etc. (Ever tried washing your hair with one hand, or one tied into a garbage bag?)

Likewise, the leg casing will provide for more normal bathing activities, allowing the wearer to step into the water before sitting in the tub, although complete submersion is not recommended, based on the fit of the elastic on the individual. The mitten alternative would be provided for pediatric wearers.

This product is primarily made for, but not limited to medical uses, such as coverings for casts, sutures, burns, IVs, cuts, scabs, pins, birthmarks, open wounds and all other surface related problems that would require the location of concern to be kept free from moisture or other contaminants.

The dimensions of the products are as follows:

The full arm glove is 34 inches in total length. The opening is 19 inches before elastic.

Above elbow total length is 25 inches and 16 inches at the opening before elastic.

Above wrist total length is 14 inches and 12½ inches at the opening before elastic.

The hand part of the glove will consist of five individual extensions for the fingers and thumb. * The dimensions are 3 inches in length for the thumb, 3½ inches in length for the forefinger and ring finger, 4 inches in length for the middle finger and 2½ inches in length for the pinky finger. Each finger section is 1¾ inches across at the base, the thumb section is 2½ inches across at the base and will be tapered the length of each section to a rounded end.

Mitten lengths used for pediatric wearers are:

25 inches for full arm length from tip of mitten to opening which is 16 inches before elastic.

18 inches for above elbow length from tip of mitten to opening which is 14½ inches before elastic.

14 inches for above wrist length from tip of mitten to opening which is 12½ inches before elastic.

Each mitten will accomodate a section for the thumb and another for the four fingers.

Leg injury tube protectors will be:

40 inches for full leg length with 32 inch opening before elastic.

32 inches for above knee length with 25 inch opening before elastic.

15 inches for above ankle length with 20 inch opening before elastic.

The sealed rounded end of the tube is 15 inches in circumference. (7½ inches in diameter).

Smaller tubes sizes will be added for infants which will be used for either hand/arm or foot/leg.

These dimensions may vary according to the manufacturing and materials requirements.

Dimensions can be provided if required.

What I claim as my invention is the disposable, flexible plastic, water resistent limb injury protector:

1. This product is made out of dual clear plastic polyethylene or polypropylene film cut by a heated die into a sealed sleeve-like covering, ending in a fingered glove, or mitten for the hand and arm or rounded tube for the foot and leg, elasticized at the opening to provide comfortable and convenient protection of injuries of the hand/arm or foot/leg from moisture damage or contaminants encountered during daily activities, such as washing, bathing, outdoor or other activities that would require keeping the injury (or cast) free from contaminants. This elasticized sleeve product is primarily made for, but not limited to medical uses, such as coverings for casts, sutures, burns, IVs, cuts, scabs, pins, birthmarks, open wounds, and all other surface related problems that would require the location of concern to be kept free from moisture or other contaminants.
